(19) United States
(12) Patent Application Publication
(10) Pub. No.: US 2012/0141472 A1
(43) Pub. Date: Jun. 7, 2012

(54) METHODS OF SCORING GENE COPY NUMBER IN A BIOLOGICAL SAMPLE USING IN SITU HYBRIDIZATION

(76) Inventors: Shalini Singh, Tucson, AZ (US); Fabien Gaire, Oro Valley, AZ (US); James Ranger-Moore, Tucson, AZ (US)

(21) Appl. No.: 13/322,495

(22) PCT Filed: May 28, 2010
(86) PCT No.: PCT/US10/36725

§ 371 Date: Nov. 25, 2011

Related U.S. Application Data
(60) Provisional application No. 61/217,316, filed on May 29, 2009.

(51) Int. Cl. A61K 39/395; A61K 31/5377; A61K 31/517; C12Q 1/68; G01N 21/64

(52) U.S. Cl. 424/133.1; 435/6.11; 424/172.1; 514/266.4; 514/234.5

(57) ABSTRACT

Disclosed herein are methods of predicting prognosis of a neoplastic disease (such as lung cancer, for example NSCLC), including determining the IGF1R gene copy number in a biological sample from a patient having a neoplastic disease; wherein an increase in IGF1R copy number predicts a good prognosis of the neoplastic disease in the patient. Also disclosed herein are methods of scoring copy number of a gene of interest in a biological sample. The method includes identifying individual cells in the sample having highest number of signals for the gene of interest detected by in situ hybridization, counting the number of signals for the gene of interest in the identified individual cells and determining an average number of signals per cell.

### PRIORITY CLAIM

This claims the benefit of U.S. Provisional Application No. 61/217,316, filed May 29, 2009, which is incorporated herein in its entirety.

### FIELD

This disclosure relates to the field of cancer and particularly to methods for determining the prognosis of patients with cancer. This disclosure also relates to methods for scoring gene copy number detected by in situ hybridization.

### BACKGROUND

Non-small cell lung cancer (NSCLC) accounts for almost 80% of all lung cancers. This group of cancers includes adenocarcinomas, which account for approximately 50% of all cases of non-small cell lung cancer; squamous cell carcinomas, which include approximately 30% of all cases of non-small cell lung cancer; and large cell carcinomas, which account for about 10% of all non-small cell lung cancers.

Current advances in molecular cancer therapeutics provide unique opportunities to identify prognostic indicators for specific patient populations. Gene amplification and/or overexpression have been identified as an indicator of patient prognosis in a variety of tumors. For example, amplification and/or overexpression of the human epidermal growth factor receptor 2 (HER2, also known as ERBB2) tyrosine kinase is detected in 20-25% of human breast cancers. This alteration is an independent prognostic factor predictive of poor clinical outcome and a high risk of recurrence. Similarly, amplification or overexpression of the epidermal growth factor receptor (EGFR) is detected in numerous cancers (such as NSCLC, breast cancer, glioma, and head and neck cancer) and may be associated with poor prognosis. Additional markers that can predict prognosis, such as patient survival, continue to be identified.

### SUMMARY

Disclosed herein are methods of predicting prognosis of a neoplastic disease (such as lung cancer, for example NSCLC), including determining the insulin-like growth factor 1 receptor (IGF1R) gene copy number in a biological sample from a patient having a neoplastic disease; wherein an increase in IGF1R copy number predicts a good prognosis of the neoplastic disease in the patient. In some examples, an increased IGF1R copy number includes an IGF1R copy number per nucleus in the sample of greater than about two copies of the IGF1R gene per nucleus (such as greater than 2, 3, 4, 5, 10, or 20 copies). In other examples, an increased IGF1R copy number includes a ratio of IGF1R copy number to Chromosome 15 copy number in the sample of greater than about 2 (such as a ratio of greater than 2, 3, 4, 5, 10, or 20). In some method embodiments, a good prognosis is greater than 1-year survival (such as greater than 2-year survival, greater than 3-year survival, or greater than 5-year survival) of the patient after initial diagnosis of the neoplastic disease.

Other prognostic method embodiments involve detecting the IGF1R gene copy number in a biological sample from a patient having a neoplastic disease (such as lung cancer, for example NSCLC); wherein substantially no increase or a decrease in IGF1R gene copy number (such as an IGF1R gene copy number of about 2 or less) predicts a poor prognosis of the neoplastic disease in the patient.

Also disclosed herein are methods of scoring copy number of a gene of interest in a biological sample, such as gene copy number detected by an in situ hybridization assay. The method includes identifying individual cells in the sample having highest number of signals for the gene of interest detected by in situ hybridization, such that individual copies of the gene are distinguishable in cells in the sample. The number of signals for the gene is then counted in the identified individual cells and an average number of signals per cell is determined. In some examples, the biological sample includes a tumor sample (such as a breast cancer sample or a lung cancer sample). In particular examples, the number of cells identified for counting is at least 20 cells (such as at least 25, 30, 40, 50, 100, 200, 500, or 1000 cells).

In other examples the method also includes counting the number of signals for a reference (such as a chromosomal locus known not to be abnormal, for example, centromeric DNA) detected by in situ hybridization in the identified cells and determining an average ratio of the number of signals for the gene of interest to the number of signals for the reference. In particular examples, the reference and the gene of interest are on the same chromosome.

The foregoing and other features will become more apparent from the following detailed description, which proceeds with reference to the accompanying figures.

### BRIEF DESCRIPTION OF THE DRAWINGS

At least some of the following figures are submitted in color:

FIG. 1 is a histogram showing the distribution of IGF1R gene copy number in a population of NSCLC patients.

FIG. 2 is a series of photomicrographs showing dual IGF1R and chromosome 15 ISH in NSCLC adenocarcinoma (left) and squamous cell carcinoma (right).

FIG. 3 is a Kaplan-Meier plot showing progression-free survival by IGF1R copy number.

FIG. 4 is a Kaplan-Meier plot showing overall survival by IGF1R copy number.

FIG. 5A is a series of photomicrographs showing IGF1R SISH (top) and IHC (bottom) in NSCLC squamous cell carcinoma samples.

FIG. 5B is a series of photomicrographs showing IGF1R SISH (top) and IHC (bottom) in NSCLC adenocarcinoma samples.

FIG. 6 is a histogram showing IGF1R protein expression (H score) determined by IHC according to IGF1R gene copy number.

FIG. 7 is a schematic of exemplary methods of scoring gene copy number. Optional steps are enclosed in dashed lines.

### DETAILED DESCRIPTION

### I. Abbreviations

CISH: chromogenic in situ hybridization
FISH: fluorescent in situ hybridization
IGF1R: insulin-like growth factor 1 receptor
IHC: immunohistochemistry
ISH: in situ hybridization
NSCLC: non-small cell lung cancer
OS: overall survival
PFS: progression-free survival
SISH: silver in situ hybridization
TMA: tissue microarray

### II. Terms

Unless otherwise explained, all technical and scientific terms used herein have the same meaning as commonly understood by one of ordinary skill in the art to which a disclosed invention belongs. The singular terms “a,” “an,” and “the” include plural referents unless context clearly indicates otherwise. Similarly, the word “or” is intended to include “and” unless the context clearly indicates otherwise. “Comprising” means “including.” Hence “comprising A or B” means “including A” or “including B” or “including A and B.

Suitable methods and materials for the practice and/or testing of embodiments of a disclosed invention are described below. Such methods and materials are illustrative only and are not intended to be limiting. Other methods and materials similar or equivalent to those described herein can be used. For example, conventional methods well known in the art to which a disclosed invention pertains are described in various general and more specific references, including, for example, Sambrook et al., Molecular Cloning: A Laboratory Manual, 2d ed., Cold Spring Harbor Laboratory Press, 1989; Sambrook et al., Molecular Cloning: A Laboratory Manual, 3d ed., Cold Spring Harbor Press, 2001; Ausubel et al., Current Protocols in Molecular Biology, Greene Publishing Associates, 1992 (and Supplements to 2000); Ausubel et al., Short Protocols in Molecular Biology: A Compendium of Methods from Current Protocols in Molecular Biology, 4th ed., Wiley & Sons, 1999; Harlow and Lane, Antibodies: A Laboratory Manual, Cold Spring Harbor Laboratory Press, 1990; and Harlow and Lane, Using Antibodies: A Laboratory Manual, Cold Spring Harbor Laboratory Press, 1999.

All sequences associated with the GenBank Accession Nos. mentioned herein are incorporated by reference in their entirety as were present on May 29, 2009, to the extent permissible by applicable rules and/or law.

In order to facilitate review of the various embodiments of the disclosure, the following explanations of specific terms are provided:

Amplification: An increase in the amount of (number of copies of) a nucleic acid molecule, wherein the increased nucleic acid molecule is the same as or complementary to the existing nucleic acid molecule. In some examples, amplification refers to an increase in amount or copy number of a genomic (for example, chromosomal) DNA sequence. The copy number of a genomic DNA may increase (for example, in a cancer cell, such as non-small cell lung cancer cell) due to gene amplification, a process that occurs through preferential replication of a segment of a chromosome. The amplification may vary in size and in number of replicates and may encompass multiple genes.

The gene amplification may be defined in terms of a chromosomal region (such as amplification of chromosome 15q26, for example, chromosome 15q26.3) or may be defined in terms of one or more particular genes (for example amplification of the IGF1R gene). In particular examples, gene amplification refers to an increase in copy number of a chromosomal region or gene (such as chromosome 15q26.3 or IGF1R) as compared to a control cell (such as a non-tumor cell). In other examples, gene amplification refers to a particular number of copies of a gene (such as IGF1R) in a cell or nucleus, such as more than about 2.5 copies of IGF1R gene/nucleus.

Copy number: The number of copies of a nucleic acid molecule in a cell. The copy number includes the number of copies of one or more genes or portions thereof in genomic (chromosomal) DNA of a cell. In a normal cell (such as a non-tumor cell), the copy number of a gene (or any genomic DNA) is usually about two (one copy on each member of a chromosome pair). In some examples, the copy number of a gene or nucleic acid molecule includes an average copy number taken from a population of cells.

DNA (deoxyribonucleic acid): DNA is a long chain polymer which comprises the genetic material of most living organisms (some viruses have genes comprising ribonucleic acid (RNA)). The repeating units in DNA polymers are four different nucleotides, each of which comprises one of the four bases, adenine, guanine, cytosine and thymine bound to a deoxyribose sugar to which a phosphate group is attached. Triplets of nucleotides (referred to as codons) code for each amino acid in a polypeptide, or for a stop signal (termination codon). The term codon is also used for the corresponding (and complementary) sequences of three nucleotides in the mRNA into which the DNA sequence is transcribed.

Unless otherwise specified, any reference to a DNA molecule is intended to include the reverse complement of that DNA molecule. Except where single-strandedness is required by the text herein, DNA molecules, though written to depict only a single strand, encompass both strands of a double-stranded DNA molecule. Thus, a reference to the nucleic acid molecule that encodes IGF1R, or a fragment thereof, encompasses both the sense strand and its reverse complement. Thus, for instance, it is appropriate to generate probes or primers from the reverse complement sequence of the disclosed nucleic acid molecules.

Hybridization: To form base pairs between complementary regions of two strands of DNA, RNA, or between DNA and RNA, thereby forming a duplex molecule. Hybridization conditions resulting in particular degrees of stringency will vary depending upon the nature of the hybridization method and the composition and length of the hybridizing nucleic acid sequences. Generally, the temperature of hybridization and the ionic strength (such as the Na⁺ concentration) of the hybridization buffer will determine the stringency of hybridization. Calculations regarding hybridization conditions for attaining particular degrees of stringency are discussed in Sambrook et al., (1989) Molecular Cloning, second edition, Cold Spring Harbor Laboratory, Plainview, N.Y. (chapters 9 and 11) and Ausubel et al., Short Protocols in Molecular Biology: A Compendium of Methods from Current Protocols in Molecular Biology, 4th ed., Wiley & Sons, 1999.

In situ hybridization (ISH): A type of hybridization using a labeled complementary DNA or RNA strand (i.e., probe) to localize a specific DNA or RNA sequence in a portion or section of tissue (in situ), or, if the tissue is small enough (e.g., plant seeds, Drosophila embryos), in the entire tissue (whole mount ISH). This is distinct from immunohistochemistry, which localizes proteins in tissue sections. DNA ISH can be used to determine the structure of chromosomes, such as for use in medical diagnostics to assess chromosomal integrity. RNA ISH (hybridization histochemistry) is used to measure and localize mRNAs and other transcripts within tissue sections or whole mounts.

For hybridization histochemistry, sample cells and tissues are usually treated to fix the target transcripts in place and to increase access of the probe to the target molecule. As noted above, the probe is either a labeled complementary DNA or a complementary RNA (riboprobe). The probe hybridizes to the target sequence at elevated temperature, and then the excess probe is washed away (after prior hydrolysis using RNase in the case of unhybridized, excess RNA probe). Solution parameters, such as temperature, salt and/or detergent concentration, can be manipulated to remove most or all non-identical interactions (e.g. only sequences that are substantially identical or exact sequence matches will remain bound). Then, the labeled probe having been labeled effectively, such as with either radio-, fluorescent- or antigen-labeled bases (e.g., digoxigenin), is localized and potentially quantitated in the tissue using autoradiography, fluorescence microscopy or immunohistochemistry, respectively. ISH can also use two or more probes, labeled with radioactivity or non-radioactive labels, such as hapten labels, and typically differentially labeled to simultaneously detect two or more nucleic acid molecules.

Insulin-like growth factor 1 receptor (IGF1R): A tyrosine kinase receptor that binds insulin-like growth factor with high affinity. IGF1R is a heterotetramer of two extracellular α subunits and two membrane spanning β subunits which include a tyrosine kinase domain. Upon ligand binding, IGF1R becomes phosphorylated and signals via the MAP kinase and Akt/mTOR pathways.

Nucleic acid and protein sequences for IGF1R are publicly available. For example, GENBANK® Accession No. NC_{—}000015 (nucleotides 97010284-97325282) discloses an exemplary human IGF1R genomic sequence (incorporated by reference as provided by GENBANK® on May 29, 2009). In other examples, GENBANK® Accession Nos.: NM 000875, BC113610, and X04434 disclose exemplary IGF1R nucleic acid sequences, and GENBANK® Accession Nos.: NP—000866, AAI13611, and CAA28030 disclose exemplary IGF1R protein sequences, all of which are incorporated by reference as provided by GENBANK® on May 29, 2009. In certain examples, IGF1R has at least 80% sequence identity, for example at least 85%, 90%, 95%, or 98% sequence identity to a publicly available IGF1R sequence, and is an IFG1R whose copy number can predict the prognosis of a patient with neoplastic disease, such as NSCLC.

In vitro amplification: Techniques that increase the number of copies of a nucleic acid molecule in a sample or specimen by in vitro, or laboratory techniques. An example of in vitro amplification is the polymerase chain reaction, in which a biological sample collected from a subject is contacted with a pair of oligonucleotide primers, under conditions that allow for the hybridization of the primers to nucleic acid template in the sample. The primers are extended under suitable conditions, dissociated from the template, and then re-annealed, extended, and dissociated to amplify the number of copies of the nucleic acid.

The product of in vitro amplification may be characterized by electrophoresis, restriction endonuclease cleavage patterns, oligonucleotide hybridization or ligation, and/or nucleic acid sequencing, using standard techniques.

Other examples of in vitro amplification techniques include strand displacement amplification (see U.S. Pat. No. 5,744,311); transcription-free isothermal amplification (see U.S. Pat. No. 6,033,881); repair chain reaction amplification (see WO 90/01069); ligase chain reaction amplification (see EP-A-320 308); gap filling ligase chain reaction amplification (see U.S. Pat. No. 5,427,930); coupled ligase detection and PCR (see U.S. Pat. No. 6,027,889); and NASBA™ RNA transcription-free amplification (see U.S. Pat. No. 6,025,134).

Label: An agent capable of detection, for example by ELISA, spectrophotometry, flow cytometry, or microscopy. For example, a label can be attached to a nucleic acid molecule or protein (such as IGF1R nucleic acid or protein), thereby permitting detection of the nucleic acid molecule or protein. Examples of labels include, but are not limited to, radioactive isotopes, enzyme substrates, co-factors, ligands, chemiluminescent agents, fluorophores, haptens, enzymes, and combinations thereof. Methods for labeling and guidance in the choice of labels appropriate for various purposes are discussed for example in Sambrook et al. (Molecular Cloning: A Laboratory Manual, Cold Spring Harbor, N.Y., 1989) and Ausubel et al. (In Current Protocols in Molecular Biology, John Wiley & Sons, New York, 1998).

Lung cancer: A neoplastic condition of lung tissue that can be benign or malignant. The majority of lung cancers are non-small cell lung cancer (such as adenocarcinoma of the lung, squamous cell carcinoma, and large-cell cancer). Most other lung cancers are small-cell lung carcinomas. In particular examples, lung cancer includes non-small cell lung cancer.

Probe: An isolated nucleic acid molecule attached to a detectable label or reporter molecule. Typical labels include radioactive isotopes, enzyme substrates, co-factors, ligands, chemiluminescent or fluorescent agents, haptens (including, but not limited to, DNP), and enzymes. Methods for labeling and guidance in the choice of labels appropriate for various purposes are discussed, e.g., in Sambrook et al. (In Molecular Cloning: A Laboratory Manual, CSHL, New York, 1989) and Ausubel et al. (In Current Protocols in Molecular Biology, Greene Publ. Assoc. and Wiley-Intersciences, 1992).

One of ordinary skill in the art will appreciate that the specificity of a particular probe increases with its length. Thus, probes can be selected to provide a desired specificity, and may comprise at least 17, 20, 23, 25, 30, 35, 40, 45, 50 or more consecutive nucleotides of desired nucleotide sequence. In particular examples, probes can be at least 100, 250, 500, 600 or 1000 consecutive nucleic acids of a desired nucleotide sequence (such as an IGF1R gene sequence).

Prognosis: A prediction of the course of a disease, such as cancer (for example, non-small cell lung cancer). The prediction can include determining the likelihood of a subject to develop aggressive, recurrent disease, to develop one or more metastases, to survive a particular amount of time (e.g., determine the likelihood that a subject will survive 1, 2, 3, 4, or 5 years), to survive a particular amount of time without disease progression (e.g., determine the likelihood that a subject will survive 1, 2, 3, 4, or 5 years without progression), to respond to a particular therapy (e.g., chemotherapy), or combinations thereof.

Sample: A biological specimen containing genomic DNA, RNA (including mRNA), protein, or combinations thereof, obtained from a subject. Examples include, but are not limited to, peripheral blood, urine, saliva, fine needle aspirate, tissue biopsy, surgical specimen, and autopsy material. In one example, a sample includes a tumor sample (for example a NSCLC sample), such as a tumor biopsy, tumor core, lymph node tissue from a subject with a tumor, or a metastasis from a tumor. In other examples, a sample includes a control sample, such as a non-tumor cell or tissue sample.

Subject: Living multi-cellular vertebrate organisms, a category that includes human and non-human mammals.

Therapeutically effective amount: A dose sufficient to prevent advancement, delay progression, or to cause regression of a disease, or which is capable of reducing symptoms caused by the disease, such as cancer, for example lung cancer.

Tumor: A neoplasm that may be either malignant or non-malignant (benign). Tumors of the same tissue type are tumors originating in a particular organ (such as breast, prostate, bladder or lung). Tumors of the same tissue type may be divided into tumors of different sub-types (a classic example being lung tumors, which can be small cell or non-small cell tumors).

Tumors include original (primary) tumors, recurrent tumors, and metastases (secondary) tumors. A tumor recurrence is the return of a tumor, at the same site as the original (primary) tumor, after the tumor has been removed surgically, by drug or other treatment, or has otherwise disappeared. A metastasis is the spread of a tumor from one part of the body to another. Tumors formed from cells that have spread are called secondary tumors and contain cells that are like those in the original (primary) tumor. There can be a recurrence of either a primary tumor or a metastasis.

### III. Methods of Predicting Prognosis of Cancer

Methods are provided herein for determining or predicting prognosis of a neoplastic disease (such as lung cancer, for example, NSCLC) by determining IGF1R gene copy number in a biological sample obtained from a patient with a neoplastic disease. The disclosed methods include determining the copy number of the IGF1R gene in a biological sample (such as a tumor sample, for example, a NSCLC sample). In particular examples, an increase in IGF1R copy number predicts a good prognosis of the neoplastic disease in the patient. In other examples, no substantial change or a decrease in IGF1R copy number predicts a poor prognosis of the neoplastic disease in the patient.

In some examples, an increased IGF1R copy number includes IGF1R copy number per nucleus (such as average IGF1R copy number per nucleus) in the sample of greater than about two copies of the IGF1R gene per nucleus (such as greater than 2, 3, 4, 5, 10, or 20 copies). In other examples, an increased IGF1R copy number includes a ratio of IGF1R copy number to Chromosome 15 copy number (such as an average IGF1R:Chromosome 15 ratio) in the sample of greater than about 2 (such as a ratio of greater than 2, 3, 4, 5, 10, or 20).

In further examples, an increased IGF1R copy number includes an increase in IGF1R copy number relative to a control (such as an increase of about 1.5-fold, about 2-fold, about 3-fold, about 5-fold, about 10-fold, about 20-fold, or more). Therefore, in some examples, the method includes comparing the IGF1R gene copy number in the sample from the subject to the IGF1R gene copy number in a control, such as a non-neoplastic sample of the same tissue type as the neoplastic sample, or a reference value or range of values expected for IGF1R gene copy number in an appropriate normal tissue. Thus, for example, if the sample from the subject is a NSCLC sample, the control can be a normal lung sample, for example from the same subject, or a reference value representing IFG1R copy number expected in a normal lung sample. In some embodiments, the control is a sample obtained from a healthy patient or a non-tumor tissue sample obtained from a patient diagnosed with cancer. In other embodiments, the control is a historical control or standard reference value or range of values (such as a previously tested control sample, such as a group of samples that represent baseline or normal values, such as the IGF1R gene copy number in non-tumor tissue).

In other examples, no substantial change or a decrease in IGF1R gene copy number includes IGF1R copy number per nucleus (such as average IGF1R copy number per nucleus) in the sample of about two or less copies of the IGF1R gene per nucleus (such as less than 2, 1.5, or 1 copies). In other examples, no substantial change or a decrease in IGF1R copy number includes a ratio of IGF1R copy number to Chromosome 15 copy number (such as an average IGF1R:Chromosome 15 ratio) in the sample of about 2 or less (such as a ratio of less than 2, 1.5, or 1). In further examples, no substantial change or a decrease in IGF1R copy number includes no substantial increase or a decrease in IGF1R copy number relative to a control.

Prognosis for a subject can be characterized by any parameter known in the art, including, for instance, actual survival after initial diagnosis (such as 6-month survival, 1-year survival, 2-year survival, or 5-year survival), and/or actual survival relative to the average survival for similarly situated patients. A good prognosis entails, e.g., survival of a patient for more than 1 year after initial diagnosis (such as more than 2 years or more than 5 years), or survival of a patient for more than 6 months longer (e.g., more than 1 year longer, more than 2 years longer, more than 5 years longer) than the average survival for similarly situated patients. A poor prognosis entails, e.g., survival of a patient for less than 5 years after initial diagnosis (such as less than 2 years or less than 1 year), or survival of a patient less than the average survival for similarly situated patients (such as, about 3 months less than average survival, about 6 months less than average survival, or about 1 year less than average survival).

In other examples, a good prognosis further predicts that a neoplasm may be less aggressive (e.g., less rapidly growing, and/or less likely to metastasize). A good prognosis may entail progression-free survival (such as lack of recurrence of the primary tumor or lack of metastasis) of a patient for more than 1 year after initial diagnosis (such as more than 2 years or more than 5 years), or progression-free survival of a patient for more than 6 months longer (e.g., more than 1 year longer, more than 2 years longer, more than 5 years longer) than the average survival for similarly situated patients. A poor prognosis may predict that a neoplasm may be more aggressive (e.g., more rapidly growing and/or more likely to metastasize). A poor prognosis may entail, e.g., progression-free survival of a patient for less than 5 years after initial diagnosis (such as less than 2 years or less than 1 year), or progression-free survival of a patient less than the average survival for similarly situated patients (such as, about 3 months less than average survival, about 6 months less than average survival, or about 1 year less than average survival).

For example, a good prognosis includes a greater than 40% chance that the subject will survive to a specified time point (such as one, two, three, four or five years), and/or a greater than 40% chance that the tumor will not metastasize. In several examples, a good prognosis indicates that there is a greater than 50%, 60%, 70%, 80%, or 90% chance that the subject will survive and/or a greater than 50%, 60%, 70%, 80% or 90% chance that the tumor will not metastasize. Similarly, a poor prognosis includes a greater than 50% chance that the subject will not survive to a specified time point (such as one, two, three, four or five years), and/or a greater than 50% chance that the tumor will metastasize. In several examples, a poor prognosis indicates that there is a greater than 60%, 70%, 80%, or 90% chance that the subject will not survive and/or a greater than 60%, 70%, 80% or 90% chance that the tumor will metastasize.

Methods of determining the copy number of a gene or chromosomal region are well known to those of skill in the art. In some examples, the methods include in situ hybridization (such as fluorescent, chromogenic, or silver in situ hybridization), comparative genomic hybridization, or polymerase chain reaction (such as real-time quantitative PCR). Exemplary methods are discussed in more detail below.

A. Biological Samples

Exemplary samples include, without limitation, blood smears, cytocentrifuge preparations, cytology smears, core biopsies, fine-needle aspirates, and/or tissue sections (e.g., cryostat tissue sections and/or paraffin-embedded tissue sections). Methods of obtaining a biological sample from a subject are known in the art. For example, methods of obtaining lung tissue or lung cells are routine. Exemplary biological samples may be isolated from normal cells or tissues, or from neoplastic cells or tissues. Neoplasia is a biological condition in which one or more cells have undergone characteristic anaplasia with loss of differentiation, increased rate of growth, invasion of surrounding tissue, and which cells may be capable of metastasis. In particular examples, a biological sample includes a tumor sample, such as a sample containing neoplastic cells.

Exemplary neoplastic cells or tissues may be isolated from solid tumors, including lung cancer (e.g., non-small cell lung cancer, such as lung squamous cell carcinoma), breast carcinomas (e.g. lobular and duct carcinomas), adrenocortical cancer, ameloblastoma, ampullary cancer, bladder cancer, bone cancer, cervical cancer, cholangioma, colorectal cancer, endometrial cancer, esophageal cancer, gastric cancer, glioma, granular call tumor, head and neck cancer, hepatocellular cancer, hydatiform mole, lymphoma, melanoma, mesothelioma, myeloma, neuroblastoma, oral cancer, osteochondroma, osteosarcoma, ovarian cancer, pancreatic cancer, pilomatricoma, prostate cancer, renal cell cancer, salivary gland tumor, soft tissue tumors, Spitz nevus, squamous cell cancer, teratoid cancer, and thyroid cancer.

For example, a sample from a tumor that contains cellular material can be obtained by surgical excision of all or part of the tumor, by collecting a fine needle aspirate from the tumor, as well as other methods known in the art. In particular examples, a tissue or cell sample is applied to a substrate and analyzed to determine IGF1R gene copy number. A solid support useful in a disclosed method need only bear the biological sample and, optionally, but advantageously, permit the convenient detection of components (e.g., proteins and/or nucleic acid sequences) in the sample. Exemplary supports include microscope slides (e.g., glass microscope slides or plastic microscope slides), coverslips (e.g., glass coverslips or plastic coverslips), tissue culture dishes, multi-well plates, membranes (e.g., nitrocellulose or polyvinylidene fluoride (PVDF)) or BIACORE™ chips. In particular examples, a NSCLC sample obtained from a subject is analyzed to determine the IGF1R gene copy number.

The samples described herein can be prepared using any method now known or hereafter developed in the art. Generally, tissue samples are prepared by fixing and embedding the tissue in a medium. In other examples, samples include a cell suspension which is prepared as a monolayer on a solid support (such as a glass slide) for example by smearing or centrifuging cells onto the solid support. In further examples, fresh frozen (for example, unfixed) tissue sections may be used in the methods disclosed herein.

In some examples an embedding medium is used. An embedding medium is an inert material in which tissues and/or cells are embedded to help preserve them for future analysis. Embedding also enables tissue samples to be sliced into thin sections. Embedding media include paraffin, celloidin, OCT™ compound, agar, plastics, or acrylics.

Many embedding media are hydrophobic; therefore, the inert material may need to be removed prior to histological or cytological analysis, which utilizes primarily hydrophilic reagents. The term deparaffinization or dewaxing is broadly used herein to refer to the partial or complete removal of any type of embedding medium from a biological sample. For example, paraffin-embedded tissue sections are dewaxed by passage through organic solvents, such as toluene, xylene, limonene, or other suitable solvents.

The process of fixing a sample can vary. Fixing a tissue sample preserves cells and tissue constituents in as close to a life-like state as possible and allows them to undergo preparative procedures without significant change. Fixation arrests the autolysis and bacterial decomposition processes that begin upon cell death, and stabilizes the cellular and tissue constituents so that they withstand the subsequent stages of tissue processing, such as for ISH.

Tissues can be fixed by any suitable process, including perfusion or by submersion in a fixative. Fixatives can be classified as cross-linking agents (such as aldehydes, e.g., formaldehyde, paraformaldehyde, and glutaraldehyde, as well as non-aldehyde cross-linking agents), oxidizing agents (e.g., metallic ions and complexes, such as osmium tetroxide and chromic acid), protein-denaturing agents (e.g., acetic acid, methanol, and ethanol), fixatives of unknown mechanism (e.g., mercuric chloride, acetone, and picric acid), combination reagents (e.g., Carnoy's fixative, methacarn, Bouin's fluid, B5 fixative, Rossman's fluid, and Gendre's fluid), microwaves, and miscellaneous fixatives (e.g., excluded volume fixation and vapor fixation). Additives may also be included in the fixative, such as buffers, detergents, tannic acid, phenol, metal salts (such as zinc chloride, zinc sulfate, and lithium salts), and lanthanum.

The most commonly used fixative in preparing samples for IHC is formaldehyde, generally in the form of a formalin solution (4% formaldehyde in a buffer solution, referred to as 10% buffered formalin). In one example, the fixative is 10% neutral buffered formalin.

### IV. Methods of Determining Gene Copy Number

The disclosed methods include determining the copy number of the IGF1R gene in a biological sample (such as a tumor sample, for example, a NSCLC sample). If the sample has increased IGF1R gene copy number relative to a control (such as an increase of about 1.5-fold, 2-fold, 3-fold, 5-fold, 10-fold, 20-fold, or more relative to a normal non-neoplastic sample or reference value), or alternatively, if the copy number of the IGF1R gene is greater than about two (such as greater than about 2, 3, 4, 5, 10, 20, or more) or the ratio of IGF1R gene copy number to Chromosome 15 copy number is greater than about two (such as greater than about 2, 3, 4, 5, 10, 20, or more), the sample is considered to have an increased IGF1R gene copy number, and thus a good prognosis. Conversely, if the sample has no change or a decrease in IGF1R gene copy number relative to a control (such as a normal non-neoplastic sample or reference value), or if the IGF1R gene copy number is less than about two or the ratio of IGF1R gene copy number to Chromosome 15 copy number is less than about two, the subject has a poor prognosis.

Methods of determining the copy number of a gene or chromosomal region are well known to those of skill in the art. In some examples, the methods include in situ hybridization (such as fluorescent, chromogenic, or silver in situ hybridization), comparative genomic hybridization, or polymerase chain reaction (such as real-time quantitative PCR).

In particular examples, IGF1R gene copy number is determined by in situ hybridization (ISH), such as fluorescence in situ hybridization (FISH), chromogenic in situ hybridization (CISH), or silver in situ hybridization (SISH). For example, using FISH, a DNA probe (such as an IGF1R probe) is labeled with a fluorescent dye or a hapten (usually in the form of fluor-dUTP or hapten-dUTP that is incorporated into the DNA using enzymatic reactions, such as nick translation or PCR). The labeled probe is hybridized to chromosomes or nuclei on slides under appropriate conditions. After hybridization, the labeled chromosomes or nuclei are visualized either directly (in the case of a fluor-labeled probe) or indirectly (using fluorescently labeled anti-hapten antibodies to detect a hapten-labeled probe). In the case of CISH, the probe is labeled with a hapten (such as digoxigenin, biotin, or fluorescein) and hybridized to chromosome or nuclear preparations under appropriate conditions. The probe is detected with an anti-hapten antibody, which is either conjugated to an enzyme (such as horseradish peroxidase or alkaline phosphatase) that produces a colored product at the site of the hybridized probe in the presence of an appropriate substrate (such as DAB, NBT/BCIP, etc.), or with a secondary antibody conjugated to the enzyme. SISH is similar to CISH, except that the enzyme (such as horseradish peroxidase) conjugated to the antibody (either anti-hapten antibody or a secondary antibody) catalyzes deposition of metal nanoparticles (such as silver or gold) at the site of the hybridized probe. For ISH methods, IGF1R copy number may be determined by counting the number of fluorescent, colored, or silver spots on the chromosome or nucleus.

In some examples, the number of spots per cells is distinguishable in the identified cells and the number of spots are counted (or enumerated) and recorded. In other examples, one or more of the identified cells may include a cluster, which is the presence of multiple overlapping signals in a nucleus that cannot be counted (or enumerated). In particular examples, the number of copies of the gene (or chromosome) may be estimated by the person (or computer, in the case of an automated method) scoring the slide. For example, one of skill in the art of pathology may estimate that a cluster contains a particular number of copies of a gene (such as 10, 20, or more copies) based on experience in enumerating gene copy number in a sample. In other examples, the presence of a cluster may be noted as a cluster, without estimating the number of copies present in the cluster.

In other examples, both the IGF1R gene and Chromosome 15 DNA (such as Chromosome 15 centromeric DNA) are detected in a sample from the subject, for example by ISH. Chromosome 15-specific probes are well known in the art and include commercially available probes, such as Vysis CEP 15 (D15Z1) probe (Abbott Molecular, Des Plaines, Ill.). The IGF1R gene and Chromosome 15 DNA may be detected on the same sample (for example, on a single slide or tissue section, such as a dual color assay) or in different samples from the same subject (for example, IGF1R gene is detected on one slide and Chromosome 15 DNA is detected on a matched slide from the same subject, such as a single color assay). The IGF1R and Chromosome 15 DNA are detected with two different detectable labels for dual color assay (such as two different fluorophores, two different chromogens, or a chromogen and metal nanoparticles). The IGF1R gene and Chromosome 15 DNA may be detected with the same label for single color assay. IGF1R and Chromosome 15 copy number may be determined by counting the number of fluorescent, colored, or silver spots on the chromosome or nucleus. A ratio of IGF1R gene copy number to Chromosome 15 number is then determined.

In another example, IGF1R gene copy number is determined by comparative genomic hybridization (CGH). See, e.g., Kallioniemi et al., Science 258:818-821, 1992; U.S. Pat. Nos. 5,665,549 and 5,721,098. In one example, CGH includes the following steps. DNA from tumor tissue (such as a lung cancer sample) and from normal control tissue (reference, such as a non-tumor sample) is labeled with different detectable labels, such as two different fluorophores. After mixing tumor and reference DNA along with unlabeled human Cot-1 DNA to suppress repetitive DNA sequences, the mix is hybridized to normal metaphase chromosomes. The fluorescence intensity ratio along the chromosomes is used to evaluate regions of DNA gain or loss in the tumor sample.

In a further example, IGF1R gene copy number is determined by array CGH (aCGH). See, e.g., Pinkel and Albertson, Nat. Genet. 37:S11-S17, 2005; Pinkel et al., Nat. Genet. 20:207-211, 1998; Pollack et al., Nat. Genet. 23:41-46, 1999. Similar to standard CGH, tumor and reference DNA are differentially labeled and mixed. However, for aCGH, the DNA mixture is hybridized to a slide containing hundreds or thousands of defined DNA probes (such as probes that are homologous to portions of the IGF1R gene). The fluorescence intensity ratio at each probe in the array is used to evaluate regions of DNA gain or loss in the tumor sample, which can be mapped in finer detail than CGH, based on the particular probes which exhibit altered fluorescence intensity. In one example, the array is an Agilent Human Genome CGH 44B Oligo Microarray (Agilent Technologies, Santa Clara, Calif.). In another example, the CGH array is a Whole Genome Tiling, Custom, or Chromosome specific Tiling Array (for example, a Chromosome 15 Tiling Array) as provided by Roche NimbleGen, Inc. (Madison, Wis.).

In general, CGH (and aCGH) does not provide information as to the exact number of copies of a particular genomic DNA or chromosomal region. Instead, CGH provides information on the relative copy number of one sample (such as a tumor sample, for example a lung cancer sample) compared to another (such as a control sample, for example a non-tumor cell or tissue sample). Thus, CGH is most useful to determine whether IGF1R gene copy number of a sample is increased or decreased as compared to a control sample (such as a non-tumor cell or tissue sample or a reference value).

In another example, IGF1R copy number is determined by real-time quantitative PCR (RT-qPCR). See, e.g., U.S. Pat. No. 6,180,349. In general the method measures PCR product accumulation through a dual-labeled fluorogenic probe (e.g., TAQMAN® probe). Although the PCR step can use a variety of thermostable DNA-dependent DNA polymerases, it typically employs the Taq DNA polymerase, which has a 5′-3′ nuclease activity but lacks a 3′-5′ proofreading endonuclease activity. TaqMan® PCR typically utilizes the 5′-nuclease activity of Taq or Tth polymerase to hydrolyze a hybridization probe bound to its target amplicon, but any enzyme with equivalent 5′ nuclease activity can be used. Two oligonucleotide primers are used to generate an amplicon typical of a PCR reaction. A third oligonucleotide, or probe, is designed to detect nucleotide sequence located between the two PCR primers. The probe is non-extendible by Taq DNA polymerase enzyme, and is labeled with a reporter fluorescent dye and a quencher fluorescent dye. Any laser-induced emission from the reporter dye is quenched by the quenching dye when the two dyes are located close together as they are on the probe. During the amplification reaction, the Taq DNA polymerase enzyme cleaves the probe in a template-dependent manner. The resultant probe fragments disassociate in solution, and signal from the released reporter dye is free from the quenching effect of the second fluorophore. One molecule of reporter dye is liberated for each new molecule synthesized, and detection of the unquenched reporter dye provides the basis for quantitative interpretation of the data. The DNA copy number is determined relative to a normalization gene contained within the sample, which has a known copy number (see Heid et al., Genome Research 6:986-994, 1996). Quantitative PCR is also described in U.S. Pat. No. 5,538,848. Related probes and quantitative amplification procedures are described in U.S. Pat. No. 5,716,784 and U.S. Pat. No. 5,723,591.

Additional methods that may be used to determine copy number of the IGF1R gene are known to those of skill in the art. Such methods include, but are not limited to Southern blotting, multiplex ligation-dependent probe amplification (MLPA; see, e.g., Schouten et al., Nucl. Acids Res. 30:e57, 2002), and high-density SNP genotyping arrays (see, e.g. WO 98/030883).

A person of ordinary skill in the art will appreciate that embodiments of the method disclosed herein for detection of one or more molecules, such as by in situ hybridization, can be automated. Ventana Medical Systems, Inc. is the assignee of a number of United States patents disclosing systems and methods for performing automated analyses, including U.S. Pat. Nos. 5,650,327; 5,654,200; 6,296,809; 6,352,861; 6,827,901; and 6,943,029, and U.S. published application Nos. 2003/0211630 and 2004/0052685.

### V. Method of Scoring Gene Copy Number

Also disclosed herein is a method of scoring (for example, enumerating) copy number of a gene in a sample from a subject (such as a subject with neoplastic disease), wherein the sample is stained by ISH (such as FISH, SISH, CISH, or a combination of two or more thereof) for the gene of interest and wherein individual copies of the gene are distinguishable in cells in the sample. In particular examples, the sample is a biological sample from a subject, such as a tumor sample (for example, a tumor biopsy or fine needle aspirate). Methods of determining gene copy number by ISH are well known in the art. Exemplary methods are described in Section IV (such as for determining IGF1R copy number).

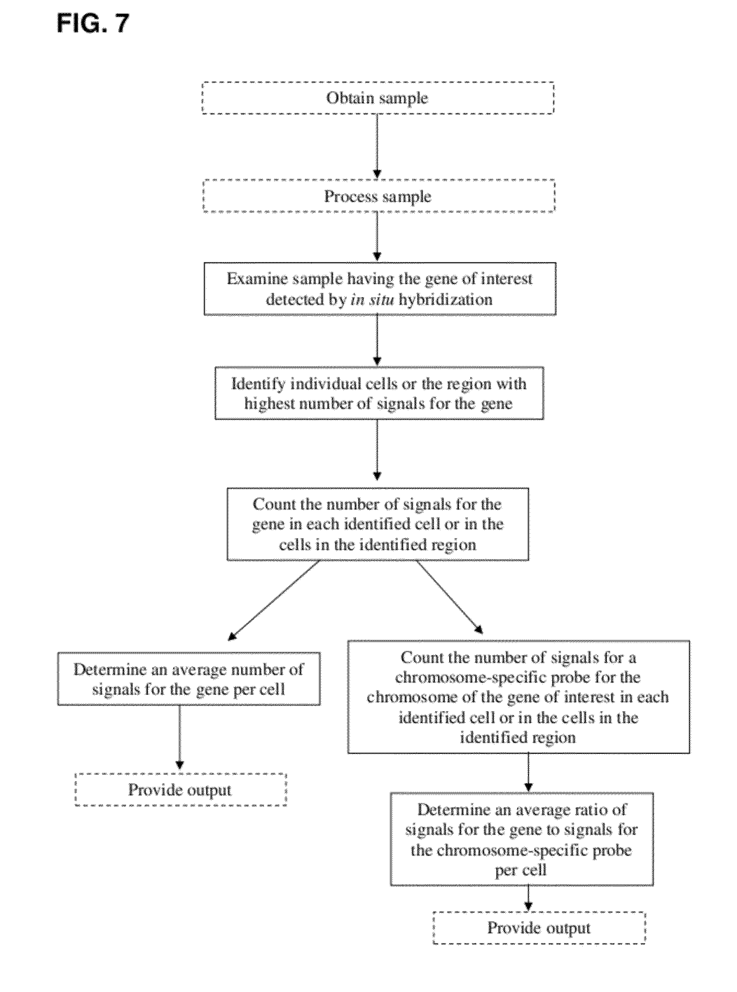

In some embodiments, the method includes identifying individual cells in a sample with the highest number of signals per nucleus for the gene (such as the strongest signal in the sample), counting the number of signals for the gene in the identified cells, and determining an average number of signals per cell, thereby scoring the gene copy number in the sample. In additional embodiments, the method further includes counting the number of signals for a reference (such as a chromosomal locus known not to be abnormal, for example, centromeric DNA) and determining an average ratio of the number of signals for the gene to the number of signals for the reference per cell. FIG. 7 provides a schematic of exemplary methods of scoring gene copy number.

The scoring method includes identifying individual cells in the sample (such as a tissue section or tumor core) having the highest number of signals (such as the highest number of spots per cell or the brightest intensity of staining) for the gene of interest in the cells in the sample. Thus, the disclosed method does not determine gene copy number in a random sampling of cells in the sample. Rather, the method includes specifically counting gene copy number in those cells that have the highest gene copy number in the sample. In some examples, identifying the individual cells having the highest number of signals for the gene includes examining a sample stained by ISH for the gene under low power microscopy (such as about 20× magnification). Cells with the strongest signal (for example, highest amplification signal under higher power) are identified for counting by eye or by an automated imaging system. In some examples, such as when the sample is a tissue section, the sample is examined (for example, visually scanned) to identify a region that has a concentration of tumor cells that has amplification of the gene. Gene copy number in the cells with highest amplification in the selected region is then counted. In other examples, such as when the sample is a tumor core (such as a tumor microarray), most of the sample is visible in the field of view under low power magnification and the individual cells (such as tumor cells) with the strongest signal (for example, highest amplification signal under high power) are separately identified for counting. In particular examples, the cells chosen for counting the gene copy number may be non-consecutive cells, such as cells that are not adjacent to or in contact with one another. In other examples, at least some of the cells chosen for counting the gene copy number may be consecutive cells, such as cells that are adjacent to or in contact with one another.

The disclosed methods include counting the number of ISH signals (such as fluorescent, colored, or silver spots) for the gene in the identified cells. The methods may also include counting the number of ISH signals (such as fluorescent, colored or silver spots) for a reference (such as a chromosome-specific probe) in the identified cells. In some examples, the number of spots per cells is distinguishable in the identified cells and the number of spots are counted (or enumerated) and recorded. In other examples, one or more of the identified cells may include a cluster, which is the presence of multiple overlapping signals in a nucleus that cannot be counted (or enumerated). In particular examples, the number of copies of the gene (or chromosome) may be estimated by the person (or computer, in the case of an automated method) scoring the slide. For example, one of skill in the art of pathology may estimate that a cluster contains a particular number of copies of a gene (such as 10, 20, or more copies) based on experience in enumerating gene copy number in a sample. In other examples, the presence of a cluster may be noted as a cluster, without estimating the number of copies present in the cluster.

The number of cells identified for counting is a sufficient number of cells that provides for detecting a change (such as an increase or decrease) in gene copy number. In some examples, the number of cells identified for counting is at least about 20, for example, at least 25, 30, 40, 50, 75, 100, 200, 500, 1000 cells, or more. In a particular example, about 50 cells are counted. In other examples, every cell in the sample or every cell in a microscope field of vision, or in a number of microscope fields (such as at least 2 microscope fields, at least 3, at least 4, at least 5, at least 6 microscope fields, and the like) which contains 3 or more copies of the gene of interest (such as 3, 4, 5, 6, 7, 8, 9, 10, 15, 20, or more) is counted.

In some examples, the biological sample is a tumor sample, such as a tumor which potentially includes a gene amplification. Exemplary biological samples include neoplastic cells or tissues, which may be isolated from solid tumors, including lung cancer (e.g., non-small cell lung cancer, such as lung squamous cell carcinoma), breast carcinomas (e.g. lobular and duct carcinomas), adrenocortical cancer, ameloblastoma, ampullary cancer, bladder cancer, bone cancer, cervical cancer, cholangioma, colorectal cancer, endometrial cancer, esophageal cancer, gastric cancer, glioma, granular call tumor, head and neck cancer, hepatocellular cancer, hydatiform mole, lymphoma, melanoma, mesothelioma, myeloma, neuroblastoma, oral cancer, osteochondroma, osteosarcoma, ovarian cancer, pancreatic cancer, pilomatricoma, prostate cancer, renal cell cancer, salivary gland tumor, soft tissue tumors, Spitz nevus, squamous cell cancer, teratoid cancer, and thyroid cancer.

In particular examples, the gene for which copy number is determined or scored is a gene which is amplified (for example, has an increased copy number, such as copy number greater than about 2), for example, in a disease state, such as cancer. Examples of genes for which copy number may be scored by the methods disclosed herein include genes which are known to be amplified in cancer (such as NSCLC, breast cancer, head and neck cancer, gastric cancer, or colorectal cancer). Examples include, but are not limited to IGF1R (15q26.3; e.g., GENBANK™ Accession No. NC_{—}000015, nucleotides 97010284-97325282), EGFR (7p12; e.g., GENBANK™ Accession No. NC_{—}000007, nucleotides 55054219-55242525), HER2 (17q21.1; e.g., GENBANK™ Accession No. NC_{—}000017, nucleotides 35097919-35138441), C-MYC (8q24.21; e.g., GENBANK™ Accession No. NC_{—}000008, nucleotides 128817498-128822856), TOP2A (17q21-q22; e.g., GENBANK™ Accession No. NC_{—}000017, complement, nucleotides 35798321-35827695), MET (7q31; e.g., GENBANK™ Accession No. NC_{—}000007, nucleotides 116099695-116225676), FGFR1 (8p11.2-p11.1; e.g., GENBANK™ Accession No. NC_{—}000008, complement, nucleotides 38387813-38445509), FGFR2 (10q26; e.g., GENBANK™ Accession No. NC_{—}000010, complement, nucleotides 123227845-123347962), MDM2 (12q14.3-q15; e.g., GENBANK™ Accession No. NC_{—}000012, nucleotides 67488247-67520481), KRAS (12p12.1; e.g. GENBANK™ Accession No. NC_{—}000012, complement, nucleotides 25249447-25295121), and TYMS (18p11.32; e.g., GENBANK™ Accession No. NC_{—}000018, nucleotides 647651-663492).

In additional examples, the method also includes scoring the number of copies per cell or nucleus of a reference, such as a chromosomal locus known not to be abnormal, for example a centromere. In some examples, the reference is on the same chromosome as the gene of interest. For example, the reference locus may be on Chromosome 15 (such as Chromosome 15 centromeric DNA) if the gene of interest is located on Chromosome 15 (such as IGF1R), the reference locus may be on Chromosome 17 if the gene of interest is located on Chromosome 17 (such as HER2 or TOP2A), and so on. Exemplary reference chromosomes that can be used for particular human genes of interest are provided in Table 1. In particular examples, the reference locus is detected by using a centromere-specific probe. Such probes are known in the art and are commercially available, for example, Vysis CEP probes (Abbott Molecular, Des Plaines, Ill.) and SPOTLIGHT centromeric probes (Invitrogen, Carlsbad, Calif.).

TABLE 1
Exemplary reference chromosomes for particular genes of interest

| Gene of Interest | Reference Chromosome |
|---|---|
| IGF1R | 15 |
| EGFR | 7 |
| HER2 | 17 |
| C-MYC | 8 |
| TOP2A | 17 |
| MET | 7 |
| FGFR1 | 8 |
| FGFR2 | 10 |
| MDM2 | 12 |
| KRAS | 12 |
| TYMS | 18 |

In additional embodiments, the method also includes obtaining a sample from the subject (for example, obtaining a tumor sample, such as a tumor biopsy or fine needle aspirate). The method may also include processing the sample, including one or more of, fixing the sample, embedding the sample, sectioning the sample, and performing ISH. In further embodiments, the method may also include providing an output (such as gene copy number or ratio of gene copy number to a reference copy number) to a user. In particular examples, the output includes, but is not limited to, reports, charts, tables, or images (for example, a representation of a field on a slide). In some examples, the output is in digital format, such as computer-readable files or records.

In some embodiments, some or all of steps of the disclosed scoring method may be performed by automation, such as by an automated microscopy system. In particular examples, an automated method may include automatically imaging label(s) bound to chromosomal sequences (such as by in situ hybridization), automatically analyzing the image for the distribution and/or intensity of the label(s), and providing a result of the analysis (such as gene copy number per cell). Such methods are known in the art, e.g., U.S. Pat. Publication Nos. 2003/0170703 and 2008/0213769; Stevens et al., J. Mol. Diagn. 9:144-150, 2007. In one example, the analysis may be performed using the Ventana Image Analysis System (VIAS, Ventana Medical Systems, Tucson, Ariz.).

In additional embodiments, the method further includes selecting a treatment for the subject, based on the gene copy number determined by the disclosed methods. The disclosed methods may further include administering the selected treatment to the subject. It is well known in the art that treatment may be selected for a subject (such as subject with a neoplastic disease) based on copy number of particular genes (such as a gene amplified in a particular cancer) in a sample from the subject. For example, if the gene for which copy number is scored is HER2, and the sample has an increased (or amplified) HER2 gene copy number, the selected therapy may include HER2 antibodies or inhibitors, such as trastuzumab, bevacizumab, or lapatinib. In other examples, if the gene for which copy number is scored is EGFR, and the sample has an increased (or amplified) EGFR gene copy number, the selected therapy may include EGFR antibodies or inhibitors, such as cetuximab, panitumumab, gefitinib, and/or erlotinib. Other treatments that may be selected and/or administered to a subject with an increase or amplification in a particular gene copy number may be selected by one of skill in the art.

The disclosure is further illustrated by the following non-limiting Examples.

### EXAMPLES

### Example 1

### IGF1R Copy Number in NSCLC

This example describes analysis of IGF1R gene copy number and prognosis in subjects with non-small cell lung carcinoma.

Tissue microarrays (TMAs) were constructed containing triplicate samples from 189 patients surgically treated for NSCLC. Characteristics of the patients are presented in Table 2. Median follow-up of the cohort was 4 years with five-year survival probability of 40% (95% CI: 31-48%). Three tissue cores of 1.5 mm diameter were obtained from different areas of primary tumor of each patient. The TMAs were created using MaxArray customized tissue microarray service (Invitrogen, South San Francisco, Calif.).

TABLE 2
Patient Characteristics

| Characteristic | N (%) | |
|---|---|---|
| Age | | |
| Median (Years) | 64 | |
| Range (Years) | 35-85 | |
| >60 years | 118 | (62) |
| Gender | | |
| Male | 144 | (76) |
| Female | 45 | (24) |
| Pathological Stage | | |
| I | 75 | (40) |
| II | 42 | (22) |
| III | 61 | (32) |
| IV | 8 | (4) |
| Unknown | 3 | (2) |
| Grade | | |
| G1 | 20 | (11) |
| G2 | 81 | (43) |
| G3 | 63 | (33) |
| Unknown | 25 | (13) |
| Histology | | |
| Squamous | 103 | (54) |
| Adenocarcinoma | 55 | (29) |
| Large cell | 5 | (3) |
| NSCLC/NOS | 24 | (13) |
| Other | 2 | (1) |
| Smoking | | |
| Ever | 180 | (95) |
| Never | 9 | (5) |
| Progression-free survival | | |
| Years—Median | 1.7 | |
| Overall survival | | |

| | |
|---|---|
| Years—Median | 2.3 |

An IGF1R probe was generated using the method of Farrell (International Publication No. WO 2008/028156). The probe targeted sequence between nucleotides 96869643-97413930 of human chromosome 15. This probe was used in SISH to evaluate IGF1R copy number on the TMAs. SISH was performed using ULTRAVIEW SISH kit and BENCHMARK XT automated slide processing system following the manufacturer's protocols (Ventana Medical Systems, Tucson, Ariz.). The mean number of IGF1R gene copies/nucleus/core was determined by a certified pathologist counting at least 50 representative nuclei per core. If necessary, the slides were first examined by hematoxylin and eosin staining to locate areas rich in tumor cells. The SISH stained slides were examined to confirm that the majority of cells in the area displayed a hybridization signal that was not hampered by background noise and for the presence of internal positive control adjacent to the tumor displaying one to two copies of IGF1R in normal cells. Nuclei were selected for counting IGF1R gene copies by the following criteria: 1) cells with unambiguous borders and objective interpretable signal; and 2) nuclei that were representative of the population of invasive carcinoma nuclei with the highest average number of signals, irrespective of the size of the nuclei. The number of IGF1R signals were counted in each selected nucleus. The number of signals in nuclei containing a cluster (multiple overlapping signals that cannot be enumerated) were estimated by the pathologist.

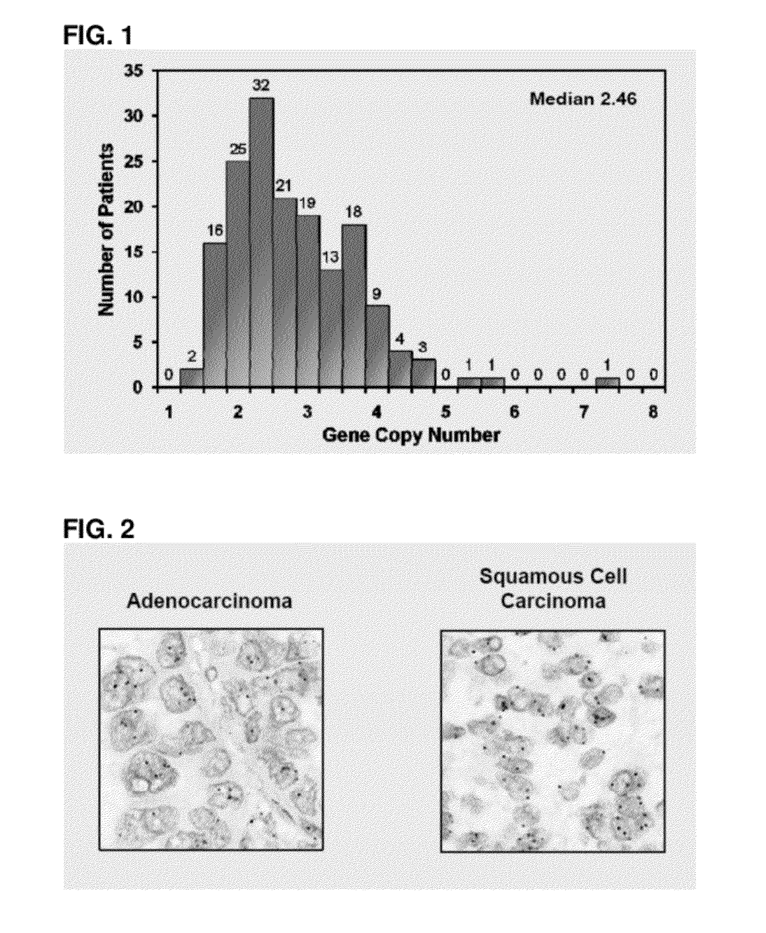

The median IGF1R copy number/nucleus was 2.46 (FIG. 1). The core with the highest mean IGF1R copy number/nucleus from each patient was used for all subsequent models. The association of IGF1R copy number with the patient characteristics was analyzed (Table 3). IGF1R copy number of >2.46/nucleus was significantly associated with squamous cell carcinoma. IGF1R and chromosome 15 copy number were also analyzed by dual ISH. IGF1R was detected using SISH, as described above. Chromosome 15 was detected using a chromosome 15 centromeric probe (Choo et al., Genomics 7:143-151, 1990) and CISH. As shown in FIG. 2, copies of IGF1R and chromosome 15 could be distinguished, making it possible to determine an IGF1R/chromosome 15 ratio.

TABLE 3
Gene copy number vs. patient characteristics

| | IGF1R/ | | IGF1R/ | | |
|---|---|---|---|---|---|
| Characteristic | nucleus ≦ 2.46 | | nucleus > 2.46 | | P-value |
| Age-Years | | | | | |
| Median | 65.9 | | 62.2 | | 0.1145 |
| Range | 45-81 | | 37-85 | | |
| Gender—N (%) | | | | | |
| Male | 65 | (77) | 63 | (78) | 0.9513 |
| Female | 19 | (23) | 18 | (22) | |
| Pathological stage—N (%) | | | | | |
| I | 33 | (39) | 31 | (38) | 0.3664 |
| II | 15 | (18) | 22 | (27) | |
| III | 32 | (38) | 21 | (26) | |
| IV | 3 | (4) | 5 | (6) | |
| Unknown | 1 | (1) | 2 | (3) | |
| Grade—N (%) | | | | | |
| G1 | 10 | (12) | 10 | (12) | 0.9941 |
| G2 | 35 | (42) | 35 | (43) | |
| G3 | 27 | (32) | 26 | (32) | |
| Unknown | 12 | (14) | 10 | (12) | |
| Histology—N (%) | | | | | |
| Squamous | 40 | (47) | 53 | (65) | 0.0158 |
| Adenocarcinoma | 26 | (31) | 20 | (25) | |
| Large cell | 3 | (4) | 2 | (2) | |
| NSCLC/NOS | 15 | (18) | 4 | (5) | |
| Other | 0 | (0) | 2 | (2) | |
| Smoking—N (%) | | | | | |
| Ever | 81 | (96) | 76 | (94) | 0.4904 |
| Never | 3 | (4) | 5 | (6) | |

Cox proportional hazards models were used to model progression-free survival (PFS) and overall survival (OS) as a function of mean IGF1R copy number, providing the primary evidence for prognostic utility. The Cox models also contained the following variables as statistical controls: age, sex, smoking status (ever vs. never), tumor histology, and tumor stage. Due to data being missing on some of these variables, only 165 patients were used in the Cox models. The proportional hazards assumption of the models was tested by assessing cumulative martingale residuals plots, with the results showing that the assumption was not violated.

For PFS, the Cox model yielded an estimated hazard ratio of 0.626 (95% confidence interval of 0.471 to 0.833) with a p-value of 0.0014 (Table 4). The low p-value indicated that this finding was highly unlikely to be due to chance, supporting the prognostic value for IGF1R. Since the hazard ratio was less than one, IGF1R had a protective effect with respect to survival. Specifically, for every one-unit increase in mean IGF1R copy number, the hazard of relapsing decreased by, on average, 37.4%. This effect was above and beyond any effects of age, sex, smoking status, tumor histology, and tumor stage, since those variables were statistically controlled for in the model.

TABLE 4
Cox regression analysis - progression-free survival

| | Std | Chi- | p- | |
|---|---|---|---|---|
| Characteristic | Error | Square | value | HR (95% CI) |
| IGF1R Gene Copy | 0.1458 | 10.32 | 0.0014 | 0.626 (0.471-0.833) |
| Number | | | | |
| Age | 0.0117 | 2.65 | 0.1049 | 1.019 (0.996-1.043) |
| Male vs. Female | 0.2592 | 0.61 | 0.4344 | 1.225 (0.737-2.036) |
| Smoking: Ever vs. | 0.5039 | 0.21 | 0.6483 | 0.795 (0.296-2.134) |
| Never | | | | |
| Histology: SCC vs. | 0.3398 | 2.98 | 0.0845 | 1.798 (0.923-3.500) |
| NOS | | | | |
| Histology: AC vs. | 0.3832 | 0.04 | 0.8745 | 0.974 (0.460-2.065) |
| NOS | | | | |
| Histology: LCC vs. | 0.6572 | 1.66 | 0.2194 | 2.327 (0.641-8.449) |
| NOS | | | | |
| Stage: IA vs. IV | 0.5095 | 26.38 | <0.0001 | 0.074 (0.027-0.201) |
| Stage: IB vs. IV | 0.4587 | 24.33 | <0.0001 | 0.105 (0.043-0.259) |
| Stage: IIA vs. IV | 0.6974 | 2.05 | 0.1632 | 0.375 (0.096-1.473) |
| Stage IIB vs. IV | 0.4688 | 19.69 | 0.0001 | 0.127 (0.051-0.317) |
| Stage IIIA vs. IV | 0.4432 | 10.24 | 0.0037 | 0.246 (0.103-0.586) |
| Stage IIIB vs. IV | 0.5543 | 3.37 | 0.0772 | 0.368 (0.124-1.090) |

For OS, the results were similar to PFS, with the Cox model yielding an estimated hazard ratio of 0.644 (95% confidence interval of 0.481 to 0.862) with a p-value of 0.0032 (Table 5). For every one-unit increase in mean IGF1R copy number, the hazard of dying decreased by, on average, 35.6%, above and beyond any effects of the other variables included in the model (as noted above).

TABLE 5
Cox regression analysis - overall survival

| | Std | Chi- | p- | |
|---|---|---|---|---|
| Characteristic | Error | Square | value | HR (95% CI) |
| IGF1R Gene Copy | 0.1491 | 8.73 | 0.0032 | 0.644 (0.481-0.862) |
| Number | | | | |
| Age | 0.0120 | 1.58 | 0.2095 | 1.015 (0.992-1.039) |
| Male vs. Female | 0.2831 | 2.39 | 0.1226 | 1.552 (0.891-2.704) |
| Smoking: Ever vs. | 0.5071 | 1.70 | 0.1929 | 0.514 (0.190-1.389) |
| Never | | | | |
| Histology: SCC vs. | 0.3437 | 1.45 | 0.2421 | 1.519 (0.774-2.983) |
| NOS | | | | |
| Histology: AC vs. | 0.3978 | 0.70 | 0.4264 | 0.733 (0.337-1.598) |
| NOS | | | | |
| Histology: LCC vs. | 0.6687 | 0.09 | 0.8284 | 1.157 (0.312-4.294) |
| NOS | | | | |
| Stage: IA vs. IV | 0.5209 | 24.58 | <0.0001 | 0.078 (0.028-0.218) |
| Stage: IB vs. IV | 0.4582 | 21.98 | <0.0001 | 0.122 (0.050-0.299) |
| Stage: IIA vs. IV | 0.8060 | 3.29 | 0.0735 | 0.242 (0.050-1.171) |
| Stage IIB vs. IV | 0.4714 | 18.09 | 0.0001 | 0.140 (0.056-0.353) |
| Stage IIIA vs. IV | 0.4451 | 7.95 | 0.0077 | 0.298 (0.124-0.713) |
| Stage IIIB vs. IV | 0.5348 | 0.48 | 0.5162 | 0.737 (0.258-2.108) |

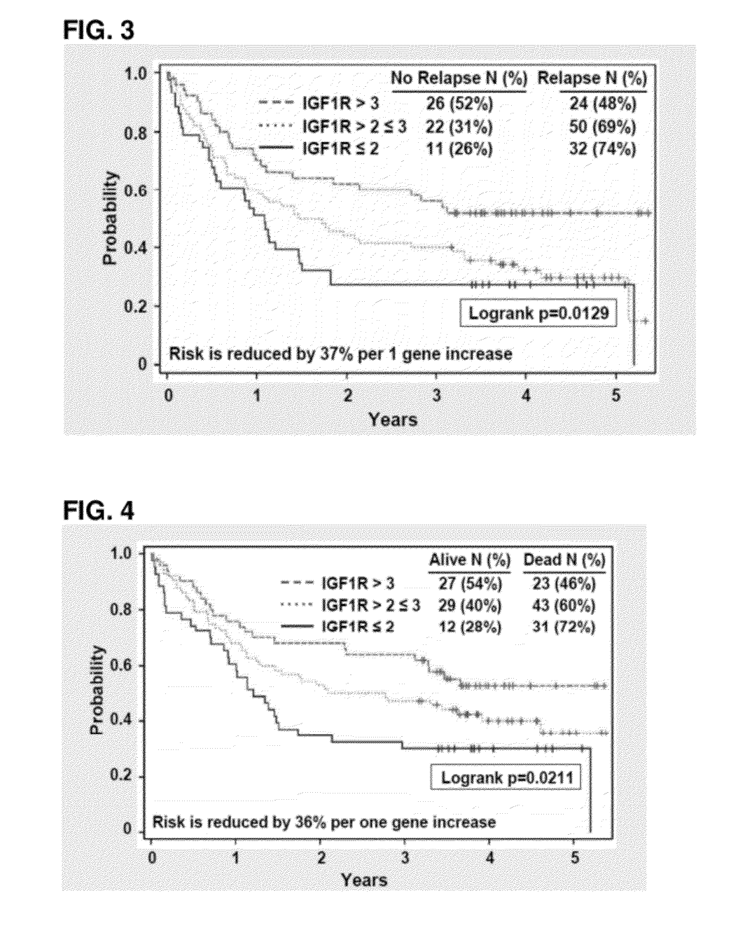

For both survival outcomes, the Kaplan-Meier plots were consistent with and supportive of the results obtained by the Cox models (FIGS. 3 and 4). For each outcome, Kaplan-Meier survival functions were constructed, stratified by three levels of mean IGF1R copy number: (a)≦2, (b)>2 and ≦3, and (c)>3 copies per nucleus. In both cases, a monotonic relationship was observed, with higher mean IGF1R copy number resulting in greater probability of survival at any given time point. In both cases, these differences were statistically significant at p<0.05 using the logrank test.

Immunohistochemistry evaluation of the TMAs was done using the Ventana G11 anti-IGF1R antibody (CONFIRM anti-IGF1R antibody, Ventana Medical Systems, Tucson, Ariz.). Staining was performed according to the manufacturer's protocol using BENCHMARK XT and the ULTRAVIEW-DAB detection kit (Ventana Medical Systems, Tucson, Ariz.). Samples were incubated with the primary antibody for 16 minutes. For all assays, scoring was based on assessment of staining intensity (0-4) and the percentage of positive cells (0-100%). Each intensity level was multiplied by the percentage of positive cells displaying that intensity and all values were added to obtain a final IHC (H) score (total score range: 0-400). For each patient, the core with the highest H score was utilized for final analysis.

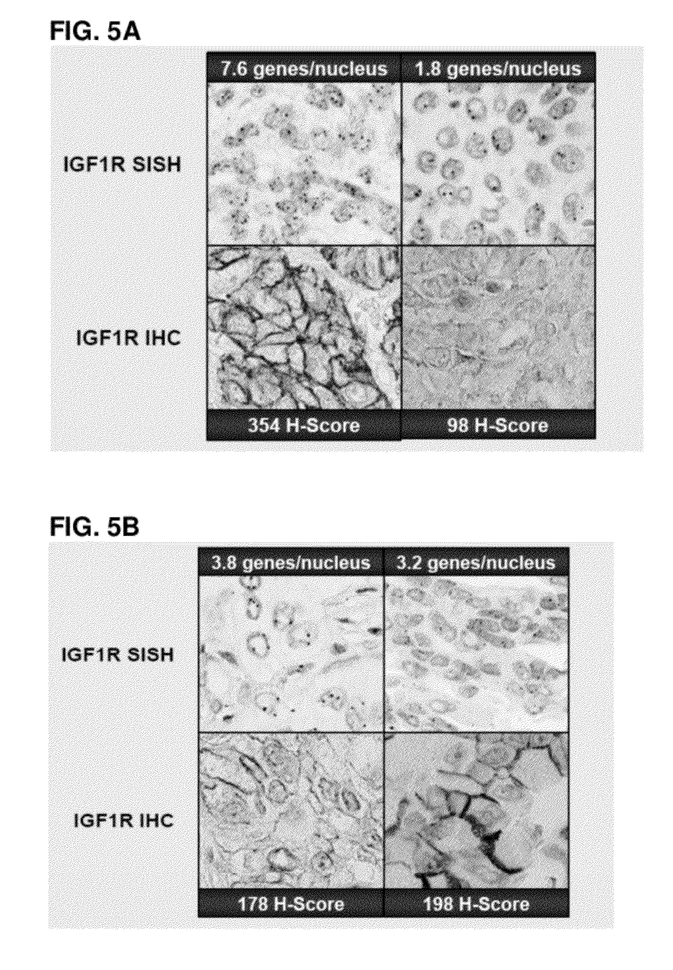

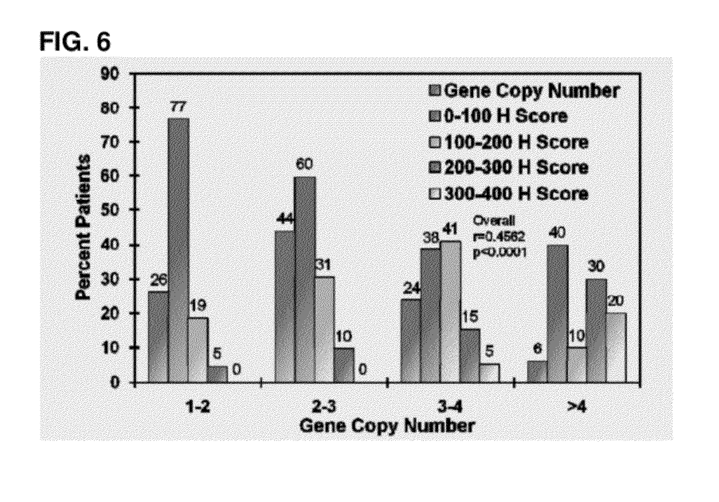

Examples of SISH analysis of IGF1R copy number and IGF1R IHC staining and scores are shown for squamous cell carcinoma (FIG. 5A) and adenocarcinoma (FIG. 5B) samples. The IHC scores generally correlated with the IGF1R gene copy number (FIG. 6).

### Example 2

### Determining Prognosis of a Subject with Cancer

This example describes particular methods that can be used to determine a prognosis of a subject diagnosed with cancer. However, one skilled in the art will appreciate that methods that deviate from these specific methods can also be used to successfully provide the prognosis of a subject with cancer.

A tumor sample (such as a tumor biopsy) is obtained from the mammalian subject, such as a human. Tissue samples are prepared for ISH, including deparaffinization and protease digestion. In one example, the prognosis of a tumor (for example, a lung tumor, such as a NSCLC) is determined by determining IGF1R gene copy number in a tumor sample obtained from a subject by in situ hybridization, such as SISH. For example, the sample, such as a tissue or cell sample present on a substrate (such as a microscope slide) is incubated with an IGF1R genomic probe. Hybridization of the IGF1R probe to the sample is detected, for example, using microscopy. The IGF1R gene copy number is determined by counting the number of IGF1R signals per nucleus in the sample and calculating an average IGF1R copy number/cell. An increase in IGF1R gene copy number/cell in the tumor sample (such as an IGF1R gene copy number of more than 2, 3, 4, 5, 10, 20, or more) or an increase in IGF1R gene copy number relative to a control (such as a non-neoplastic sample or a reference value) indicates a good prognosis, such as an increase in the likelihood of survival, for the subject. In contrast, no substantial change or a decrease in IGF1R gene copy number (such as an IGF1R gene copy number of about 2 or less) or no substantial change or a decrease in IGF1R gene copy number relative to a control (such as a non-neoplastic sample or a reference value) indicates a poor prognosis, such as a decrease in the likelihood of survival, for the subject.

In another example, the prognosis of a tumor (for example, a lung tumor, such as a NSCLC) is determined by determining a ratio of IGF1R gene copy number to Chromosome 15 centromere copy number in a tumor sample obtained from a subject by in situ hybridization, such as SISH. For example, the sample, such as a tissue or cell sample present on a substrate (such as a microscope slide) is incubated with an IGF1R genomic probe. The same sample or a matched sample from the same subject is incubated with a Chromosome 15 centromere probe. Hybridization of the IGF1R probe to the sample is detected, for example, using microscopy. Hybridization of the Chromosome 15 centromere probe is also detected, for example, using microscopy. The IGF1R gene copy number is determined by counting the number of IGF1R probe signals per nucleus in the sample and the Chromosome 15 centromere number is determined by counting the number of Chromosome 15 centromere probe signals per nucleus in the sample. An increase in the ratio of IGF1R gene copy number to Chromosome 15 centromere copy number per cell in the tumor sample (such as a ratio of more than 2, 3, 4, 5, 10, 20, or more) or an increase in the ratio of IGF1R gene copy number to Chromosome 15 centromere copy number relative to a control (such as a non-neoplastic sample or a reference value) indicates a good prognosis, such as an increase in the likelihood of survival, for the subject. In contrast, no substantial change or a decrease in the ratio of IGF1R gene copy number to Chromosome 15 centromere copy number (such as an IGF1R gene copy number to Chromosome 15 copy number ratio of about 2 or less) or no substantial change or a decrease in the ratio of IGF1R gene copy number to Chromosome 15 centromere copy number relative to a control (such as a non-neoplastic sample or a reference value) indicates a poor prognosis, such as a decrease in the likelihood of survival, for the subject.

### Example 3

### Enumeration of Gene Copy Number

This example describes an exemplary method of scoring copy number of a gene of interest detected by in situ hybridization.

A sample is obtained that has been stained by in situ hybridization for the gene of interest. The sample may be a tumor sample, such as a tumor that potentially has an amplification in the gene of interest, such as a breast tumor, lung tumor (for example, a NSCLC tumor), ovarian tumor, gastric tumor, esophageal tumor, or head and neck tumor.

The sample is examined by microscopy (such as brightfield microscopy if the sample has been stained using CISH or SISH, or fluorescence microscopy if the sample has been stained using FISH). The cells in the sample that have the highest number of signals (such as the highest number of spots per cell or the brightest intensity of staining) for the gene of interest are identified. The number of signals is counted in each identified cell until a pre-determined number of cells have been counted (such as about 20, 25, 30, 40, 50, 100, 200, 500, 1000 cells or more). The number of signals is divided by the number of cells counted, determining an average number of signals per cell, which is the average gene copy number. The resulting gene copy number can then be outputted or provided to a user.

In some examples, the sample has also been stained by in situ hybridization for a reference, such as a chromosomal locus which is known not to be abnormal, for example centromeric DNA. The number of signals for the reference is counted in the same cells as were identified and counted for the gene of interest. The number of signals of the gene of interest is divided by the number of signals of the reference, determining an average ratio of gene:reference per cell. The resulting ratio of gene copy number to reference copy number can then be outputted or provided to a user.

In view of the many possible embodiments to which the principles of the disclosure may be applied, it should be recognized that the illustrated embodiments are only examples and should not be taken as limiting the scope of the invention. Rather, the scope of the invention is defined by the following claims. We therefore claim as our invention all that comes within the scope and spirit of these claims.

1. A method of scoring copy number of a gene of interest in a biological sample from a subject, comprising:
identifying individual cells in the sample which have highest number of signals for the gene detected by in situ hybridization, wherein individual copies of the gene are distinguishable in cells in the sample;
counting a number of signals for the gene in each of the identified cells; and
determining an average number of signals per cell in the identified cells, thereby scoring the gene copy number.

2. The method of claim 1, further comprising:
counting a number of signals for a reference detected by in situ hybridization in the identified cells, wherein individual copies of the reference are distinguishable in cells in the sample; and
determining an average ratio of the number of signals for the gene to the number of signals for the reference in the identified cells.

3. The method of claim 2, wherein the reference and the gene of interest are on the same chromosome.

4. The method of claim 3, wherein the reference is centromere DNA.

5. The method of claim 1, wherein the in situ hybridization comprises silver in situ hybridization, chromogenic in situ hybridization, fluorescent in situ hybridization, or a combination of two or more thereof.

6. The method of claim 1, wherein the biological sample comprises a tumor sample.

7. The method of claim 6, wherein the tumor sample comprises a lung tumor, breast tumor, ovarian tumor, gastric tumor, head and neck tumor, esophageal tumor, or glioma.

8. The method of claim 1, wherein the number of signals is counted in at least 20 cells.

9. The method of claim 8, wherein the number of signals is counted in at least 50 cells.

10. The method of claim 9, wherein the number of signals is counted in at least 200 cells.

11. The method of claim 1, wherein the gene of interest is IGF1R, HER2, EGFR, MET, TOP2A, or MYC.

12. The method of claim 1, wherein the counting is performed by an automated imaging system.

13. The method of claim 1, further comprising:
selecting a treatment for the subject based on the gene copy number.

14. The method of claim 13, further comprising:
administering the selected treatment to the subject.

15. The method of claim 1, further comprising:
obtaining the sample;
processing the sample for in situ hybridization.

16. The method of claim 1, further comprising:
providing the gene copy number to a user.

17. The method of claim 1, further comprising:
scanning the biological sample; and
identifying at least three regions that have the highest concentration of gene signal, wherein the identifying the individual cells in the sample which have the highest number of signals for the gene detected by in situ hybridization is performed in the at least three regions.
